(19) United States
(12) Patent Application Publication
(10) Pub. No.: US 2007/0156046 A1
(43) Pub. Date: Jul. 5, 2007

(54) Method of enriching hyperpolarized atom nuclei and an apparatus for implementing the method

(76) Inventors: Friedrich Wolfgang Hasing, Julich (DE); Stephan Appelt, Julich (DE); Kerstin Munnemann, Aachen (DE)

Correspondence Address: THE FIRM OF KARL F ROSS, 5676 RIVERDALE AVENUE, PO BOX 900, RIVERDALE (BRONX), NY, 10471-0900, US

(21) Appl. No.: 10/586,569

(22) PCT Filed: Dec. 8, 2004
(86) PCT No.: PCT/DE04/02689

§ 371 Date: Jul. 18, 2006

(30) Foreign Application Priority Data
Jan. 19, 2004 (DE) 2004 002 639.4

(51) Int. Cl. A61B 5/05

(52) U.S. Cl. 600/420

(57) ABSTRACT

The invention relates to a method for enriching hyperpolarized nuclei. According to the inventive method, hyperpolarized atomic nuclei flowing in a gaseous mixture are dissolved in a solvent which is cooled at less than 293 K. A device for carrying out the method comprises at least one chamber with means for the degassing of hyperpolarized atomic nuclei which are dissolved in a solvent which is located in the chamber. The device comprises means for forming a magnetic field of 0.04 T maximum. Die Erfindung betrifft ein Verfahren zur Anreicherung von hyperpolarisienen Atomkernen. Das Ver-fahren sieht vor, in einem Gasgemisch stromende hyperpolarisierte Atomkeme in einem unterhalb 293 K gekühlten Lösungsmittel zu lösen. Eine Vorrichtung zur Durchführung des Verfahrens umfasst mindestens eine Kammer mit Mitteln zur Entgasung von hyperpolarisiente Atomkemen, die in einem in der Kammer befindlichen Lösungsmittel gelöst sind. Die Vorrichtung umfasst Mittel zur Ausbildung eines Magnetfeldes von maximal 0,04 T.

The invention relates to a method of enriching hyperpolarized atomic nuclei and an apparatus for carrying out the method.

Recent developments in magnetic resonance tomography (MRT) and magnetic resonance spectroscopy (NMR) with polarized noble gases anticipate applications in medicine, physics and materials science. Large polarizations of nuclear spins of noble gases may be achieved by optical pumping using alkali atoms, as described in the publication Happer, et al., Phys. Rev. A, 29, 3092 (1984).

Through optical pumping by incident light radiation into matter, the occupation numbers of certain energy states are increased significantly relative to the state of equilibrium. Polarization thereby refers to the degree of orientation (ordering) of the spins of atomic nuclei, or electrons. For instance, 100 percent polarization means that all nuclei or electrons are oriented likewise. Polarization of nuclei or electrons is tied to a magnetic moment.

Hyperpolarized ¹²⁹Xe is, for instance, inhaled by or injected into a human being. The polarized xenon accumulates 10 to 15 seconds later in the brain. The distribution of the noble gas in the brain is determined by using magnetic resonance tomography. The result is used for further analysis.

Choosing a polarized noble gas depends in each case on its actual application, ¹²⁹Xe has a great chemical shift. If, for example, xenon is adsorbed on a surface, its resonance frequency changes significantly. Moreover, xenon is soluble in lipophilic liquids. When such properties is desired, xenon is used.

The noble gas helium is hardly soluble in liquids. The isotope ³He is therefore regularly used, when cavities are concerned. The human lung is an example of such a cavity.

Some noble gases exhibit other useful properties. The isotopes ⁸³Kr, ²¹Ne and ¹³¹Xe have a quadruple moment that is of interest, e.g., for experiments in basic research or surface physics. However, these noble gases are very expensive, which makes them unsuitable for applications, in which greater amounts are used.

From the publication Driehuys et al. (Appl. Phys. Lett. (1996). 69, 1668) it is known how to polarize .¹²⁹Xe in a polarizer in the following way.

Based on a gas supply, a gas flow consisting of a mixture of 1% ¹²⁹Xe and N₂, including 98% ⁴He, is mixed with Rb vapor in a container and conducted through a test cell. Using a laser, circular polarized light is produced, i.e. light in which the spin momentum or the spin of the photons has the same direction. In the test cell, the Rb atoms are optically pumped as an optically pumpable species by the laser beam (λ˜795 nm, Rb Dl line) longitudinally to a magnetic field, thereby polarizing the electron spins of the Rb atoms. The spin momentum of the photons is thereby transferred to the free electrons of the alkali atoms. The spins of the electrons of the alkali atoms vary greatly from thermal equilibrium, i.e. the alkali atoms are polarized. Through the collision of an alkali atom with a noble gas atom, the polarization of the electron spins is transferred from the alkali atom to the noble gas atom, whereby polarized noble gas forms. The polarization of the electron spin of the alkali atoms created by optical pumping of alkali atoms is thus transferred through spin exchange of the alkali electron to the nuclear spin of the noble gases.

Alkali atoms are used, since they possess a large optical moment of dipole, which interacts with light. Furthermore, alkali atoms exhibit a free electron, so that unfavorable interactions between two or more electrons per atom cannot occur.

The partial pressure of ⁴He in the gas mixture is up to

bar. Compared with other partial pressures (xenon or nitrogen), this is very high. This relatively high partial pressure means that polarized atoms only rarely reach the test wall of the glass cell and lose their polarization there, e.g., through interaction with paramagnetic centers with an increasing partial pressure of ⁴He, the probability of the polarized atoms hitting the cell wall decreases.

The heavy noble gas atoms, e.g. xenon atoms, give rise to strong relaxation of the polarization of the optically pumped alkali atoms when colliding with the alkali atoms. In order to retain maximum polarization of the alkali atoms during optical pumping, the partial pressure of the xenon gas in the gas mixture must be correspondingly low. Even with a partial pressure in the gas mixture of 0.1 bar, a laser capacity of around 100 Watt is required in order to achieve a polarization of the alkali atoms of about 70 percent in the whole test volume.

For ¹²⁹Xe, the nuclear-spin polarization formation times are between 20 and 40 seconds due to the high spin exchange of the cross section. Due to the very high rubidium-spin destruction rate for rubidium xenon collisions, the xenon partial pressure may not exceed stated values, so that a sufficiently high rubidium polarization may be maintained during optical spin-exchange pumping. Hence ⁴He is employed as a buffer gas in such polarizers to achieve line broadening.

Test cells of glass are employed, blown from one piece, and in which the noble gas atoms or the atomic nuclei are optically pumped.

According to the prior art, the test cell is placed in a static magnetic field B0 of about 10 Gauss produced by coils, especially a pair of so-called Helmholtz coils. The direction of the magnetic field extends parallel to the cylinder axis of the test cell, or parallel to the direction of the laser beam. The magnetic field serves to guide the polarized atoms. The rubidium atoms, which are optically highly polarized due to the laser light, collide in the glass cell, e.g. with the xenon atoms, and release their polarization to the xenon atoms.

Typical values of partial pressure at the exit of a polarizer are P_{He} ˜7 bar, P_{N2} ˜0.07 bar, P_{Xe} ˜0.07 bar at a numerical particle density of Rb of ˜10¹⁴ cm³. With hyperpolarization of ¹²⁹Xe, its partial pressure is limited during polarization to about 0.1 bar due to spin-exchange optical pumping. In order to produce a sufficient and satisfactory amount and thickness of hyperpolarized gas for many applications, Xe thickness in the gas needs to be increased for enrichment.

A method of enriching hyperpolarized ¹²⁹Xe is disclosed in European Patent EP 0 890 066 B1. During the process, a gas mixture containing hyperpolarized ¹²⁹Xe flows through an enrichment reservoir. The reservoir is cooled, e.g. with liquid N₂ to a temperature at which xenon condenses to a frozen state, whereby it is enriched in frozen state in the reservoir from the flowing output gas. Rubidium settles on the wall at the exit of the test cell due to the high melting point compared with the melting points of the other gases. The polarized ¹²⁹Xe or the residual gas mixture is carried further by the test cell into a freezing unit, which consists of a glass flask, whose end is submerged in liquid nitrogen. The glass flask is situated in a magnetic field with a strength up to 1 Tesla. To obtain long enrichment times of ¹²⁹Xe of about 1 hour, a magnetic field must be applied on a magnitude of about 1 T, since with weaker magnetic fields and a temperature of liquid N₂, the relaxation time of the polarized Xe ice is only a few minutes, so that considerable parts of the polarization again decay for long enrichment times. Longer relaxation times (T1˜a few hours) can only be obtained, when the Xe ice is enriched/stored at a temperature of the liquid He of about 4° K.

Disadvantageously, the ¹²⁹Xe thus needs to be frozen very quickly and preferably without any loss, following polarization, by using a strong magnetic field of about 1 T, stored and subsequently re-vaporized in Xe gas. Only about 1 to 2 hours remain for using the noble gas, before the xenon polarization through relaxation has declined to such an extent that further use is no longer possible. The complexity of preparing strong magnetic fields and temperatures in order to condense ¹²⁹Xe makes this process expensive and cumbersome.

The object of the invention is to provide a method for enriching hyperpolarized atomic nuclei at a reasonable cost.

A further object of the invention is to make available an apparatus for carrying out the method.

This object is attained by a method with the totality of the features of claim 1, and by an apparatus as claimed by the dependent claims. Advantageous embodiments follow from the related claims.

The process provides for the solution of hyperpolarized atomic nuclei, flowing in a gas mixture, in a solvent cooled to below 293 K.

Solubility is defined as the density of the hyperpolarized gas in the solvent relative to the density of the hyperpolarized gas in the gas chamber situated above a given temperature and pressure. The solubility is also referred to as the Ostwald coefficient.

Within the scope of the invention, it was recognized that by using a solvent cooled below room temperature, especially an organic solvent, such as a hydrocarbon, it was possible to obtain a large increase in gas density in the solvent compared to the gas phase. In the solvent cooled below room temperature, a solubility of at least twice that at room temperature is obtained, which is used for the hyperpolarized atomic nuclei in the solvent.

The solvent below room temperature has an Ostwald coefficient of at least 2 for the hyperpolarized atomic nuclei. With decreasing temperatures, solubility or the Ostwald coefficient increases to values of up to 200. Above room temperature, the Ostwald coefficient may advantageously assume a value below 1.

The method according to the invention is by no means limited to enrichment of hyperpolarized atomic nuclei. Rather, it was found that the process will always be useful when a certain component to be enriched in a gas mixture is especially easily soluble in comparison with other components of the mixture in a solvent cooled below 93° K.

For example, the enrichment of carbon isotopes ¹²C and ¹³C from a mixture of N₂ and 02 may be mentioned. Following solution in a solvent cooled below room temperature, the carbon isotopes are enriched and separated from N₂ and 0₂. Subsequently ¹²C and ¹³C are separated through isotopic separation. Any valuable gas from a mixture may be enriched and possibly separated through further steps of the method. The method has the advantage of being economical and easy to handle.

In a further embodiment of the invention, a lipophilic solvent with high viscosity is chosen for the method.

As solvent for hyperpolarized ¹²⁹Xe, toluol with a Ostwald coefficient of about 5 is, for example, chosen as the standard conditions (293° K, 1 bar), or ethanol with an Ostwald coefficient of about 2.5 at standard conditions.

Pentane, acetone/methanol and butanol are generally suitable solvents for enrichment of hyperpolarized noble gases. The solvent may be chosen according to the temperature. In an especially advantageous embodiment of the invention, the solvent is present in liquid phase even at low temperatures, e.g. 180° K.

In a further embodiment of the invention, the melting point of the solvent decreases compared with the pure solvent without the hyperpolarized atomic nuclei.

Such a decrease of the melting point was determined within the scope of the invention in the case of toluol, in which ¹²⁹Xe was dissolved.

Dissolving hyperpolarized atomic nuclei in the solvent is thus done at temperatures that are lower than expected from prior art. This effect is used for enrichment, since lower temperatures result in a rapid increase of solubility.

As solvents, olive oil and benzol also exhibit a high Ostwald coefficient.

A suitable choice of solvent will ensure the desired increase of solubility and thus enrichment of the hyperpolarized atomic nuclei in the solvent.

The solvent comprises, for example, ethanol and/or toluol. For both solvents within the scope of the invention, a large solubility below room temperature was determined for hyperpolarized atomic nuclei. It is conceivable that these solvents may be used for enrichment of other components from a gas mixture, such as ¹³C.

In the case of hyperpolarized atomic nuclei to be enriched, their Ti relaxation times in the solvent during the process is chosen greater than their residence time in the solvent. Deuterized solvents, such as C₆D₅CD₃ (toluol) or CD₃CD₂0D (ethanol), in which the relaxation times of the hyperpolarized atomic nuclei are greater than 100 seconds, may be chosen.

Dissolving a hyperpolarized noble gas in a solvent from the gas flow of a polarizer is preferably done in a chamber, in which the solvent is either present in cooled condition or is still undergoing cooling. Similarly, other gases to be enriched are conducted into such a chamber.

The process optionally provides for introducing degassing from the solvent after dissolving a component to be enriched, e.g. a hyperpolarized noble gas. For this purpose, the solvent may be conducted out of the cooling chamber into a further chamber for degassing.

Solution and-degassing may conceivably be performed in one and the same chamber, provided the chamber has cooling and heating means.

The volume of the chambers, especially the chamber in which degassing occurs, is chosen sufficiently large, so that if hyperpolarized atomic nuclei are being enriched, the relaxation times of the nuclei through wall contact is longer at the inner walls than is the enrichment times.

The T₁ time of hyperpolarized atomic nuclei is determined, e.g. by their interaction with the inner wall of the chamber. By coating the inner walls of the chambers and/or the connection lines between the chambers, e.g. with deuterized monochlorosilane or PFA (perfluoroalkoxy compounds), T₁ times exceeding one hour may be obtained. For instance, for Xe gas, a fundamental T₁ (Xe-Xe interaction) time of 56 h/p (amagat) may be applied.

With the method according to the invention hyperpolarized atomic nuclei may therefore be enriched and stored for a longer period than with the known methods.

During the enrichment process, multiple repetition of the solution and degassing steps of the components to be enriched or the hyperpolarized atomic nuclei in and from a cooled solvent may be done in an especially advantageous manner. Especially, the solvent may be conducted in chambers especially provided for this purpose. This procedure may bring about a pump effect and for enrichment, an increased density of the components to be enriched.

The flow of the solvent may be controlled continuously or semi-continuously via the pressure in the chambers or in the pressure compensation containers or tanks.

In the case of hyperpolarized ¹²⁹Xe as a component to be enriched, the process has the advantage that depending on the solvent only minor amounts of N₂ are dissolved in the storage medium, whereas with known freezing methods, considerable amounts of N₂ are frozen.

An apparatus for carrying out the method therefore comprises at least one chamber with means for degassing the enriched components dissolved in a solvent situated in the chamber. For the purpose of degassing, the chamber is provided with, e.g. heating coils and/or means for producing ultrasound.

For a hyperpolarized noble gas as a component to be enriched, the apparatus features at least one means for forming a magnetic field with a strength not exceeding 0.04 Tesla, e.g. a Helmholtz coil.

As an advantage, expensive and heavy magnets for generating magnetic fields for the hyperpolarized atomic nuclei are thus no longer needed.

Instead, simple Helmholtz electromagnets, or permanent magnets suffice, since the cooled solvent must be exposed to a maximum magnetic field of only 0.04 Tesla for enrichment. This affects the mobility afforded by such apparatuses.

When solution and degassing occur in the same chamber, the chamber features means for degassing, and possibly cooling.

The method according to the invention enables enrichment of hyperpolarized atomic nuclei at a considerably higher temperature than is used according to prior art. The temperature of the solvent may be set to, e.g. about 180° K during enrichment, versus 77° K if condensed Xe-Sis is used. The temperature needed for the enrichment method using solvents according to the invention may thus be obtained through standard cooling methods or coolants, e.g. Peltier elements. This advantageously allows for a compact design of the apparatus according to the invention for mobile systems.

If degassing is done in another chamber, the apparatus features at least one chamber, in which the hyperpolarized atomic nuclei or other components to be enriched are dissolved, and a further, second chamber connected with this chamber. The cool solvent containing the gas to be enriched is conducted from the first to the second chamber, where degassing is done. The second chamber thereby has the said means for degassing from the solvent.

The apparatus may have a tank for the enriched gas, such as a hyperpolarized noble gas. This tank is connected with the chamber(s) in which degassing is done. The enriched, possibly hyperpolarized gas is introduced into the tank, whereas the solvent is disposed of, or returned under cooling to the cooling chamber. In an especially advantageous embodiment of the invention, the apparatus has at least two units connected successively, each consisting of a chamber, in which the component to be enriched, e.g. a hyperpolarized noble gas is dissolved in a cooled solvent, and a further chamber, in which the solvent is degassed a second time. The solution and degassing process may thus be repeated and carried out in two stages, for example. This advantageously results in a further increase in solubility or enrichment in the solvent.

The solvent cooled beneath 293° K may, of course, be used not only for enrichment, but also for storing and transporting a component, e.g. a certain hyperpolarized noble gas, to be enriched. Once enriched, gases, especially hyperpolarized noble gases, are however also of special interest in the cooled solvent, as the method according to the invention is not limited to the above-mentioned advantages. In many applications, it is necessary to make available, e.g. hyperpolarized ¹²⁹Xe pre-dissolved in a liquid in order to carry it to the objects or substances to be examined, e.g. complex molecules, or examine the diffusion of liquids in porous structures. The step involving thawing from the Xe ice is advantageously omitted in the method according to the invention. A cooled solvent containing a dissolved hyperpolarized noble gas is therefore a direct contrast agent for magnetic resonance tomographic examinations.

Cooled ethanol or toluol with dissolved ¹²⁹Xe may be also mentioned as an example.

A cool solvent containing hyperpolarized atomic nuclei, or ¹³C, 235U or 238U, whereby the solvent has a temperature of below 293 K, is therefore of special interest. The solvent may, besides toluol or ethanol, also comprise pentane or other solvents. Such a solvent may

The invention is explained in more detail in the following with reference to the examples and attached figures.

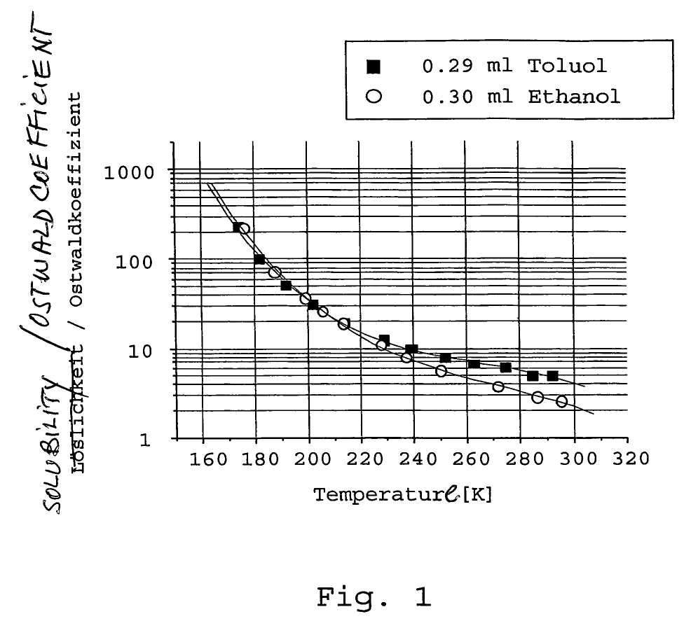

FIG. 1 shows measuring results for the solubility of ¹²⁹Xe in toluol and ethanol depending on the temperature of the solvent.

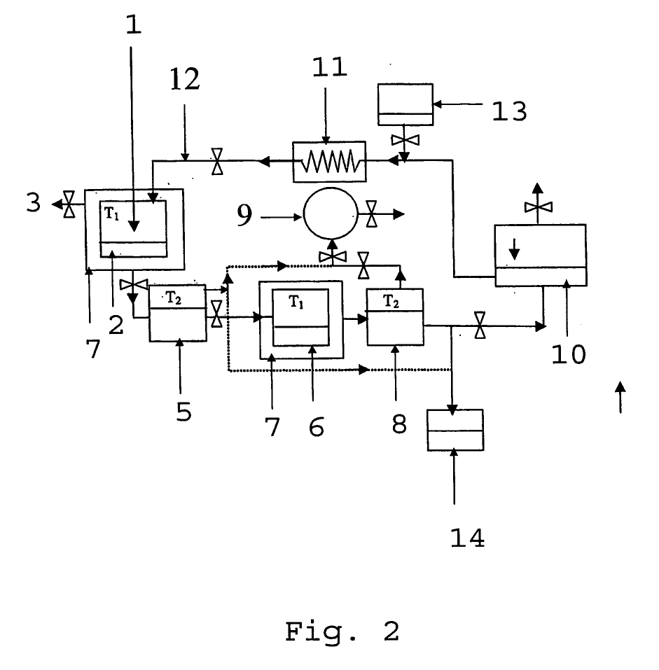

FIG. 2 shows an apparatus for carrying out the method according to the invention.

According to FIG. 1 it appeared surprisingly that already at a temperature of 273° K, a clear increase of the solubility or Ostwald coefficient of ¹²⁹Xe is obtained compared to at room temperature.

At a temperature of about 240° K, a ten times higher density of ¹²⁹Xe compared with the gas phase is already obtained both in ethanol and toluol. An even higher solubility of ¹²⁹Xe in the solvent is obtained by cooling to temperatures of 180° K, thereby obtaining a 100-fold increase of the density in both toluol and ethanol compared to the gas phase. Further cooling by just a few degrees Kelvin already results in an increase of the Ostwald coefficient to a value of 200 (FIG. 2).

A further sharp rise of solubility occurs, when the solvent is further cooled. This effect may also be produced with other solvents and hyperpolarized atomic nuclei, or with ¹³C, low temperatures is especially interesting, since with a solution of hyperpolarized atomic nuclei, a reduction of the melting point of the solvent relative to pure solvent without hyperpolarized atomic nuclei can be detected, and is also expected for other solvents.

A typical enrichment apparatus is shown in FIG. 2. A gas mixture 1 with hyperpolarized atomic nuclei flows from a polarizer (not shown) into a first chamber 2 containing a cooled solvent. Chamber 2 has means for cooling the solvent. The solvent containing the gas mixture is thereby cooled to-a temperature T₁, of, e.g. 180° K.

The gas components are enriched in chamber 2 according to their solubility in the solvent, and thus separated from one another. The unnecessary components of the mixture are released through valve 3. The solvent enriched with the hyperpolarized atomic nuclei is guided through a connection line out of chamber 2 into a further chamber 5. Chamber 5 comprises means for degassing, such as an apparatus for producing ultrasound and/or heating.

Degassing of the solvent therefore occurs as a result of heat and/or ultrasound in chamber 5.

Chamber 5 thus represents a degassing chamber 5 for the hyperpolarized atomic nuclei from the solvent. In chamber 5, a gas pressure of the originally dissolved gas arises due to the degassing of the hyperpolarized atomic nuclei via the solvent. The gas pressure is determined by the partial pressure of the hyperpolarized atomic nuclei (gas component) in solvent chamber 2, and the ratio of the solubilities of this gas component in the solvent at temperatures T₁ in chamber 2 and T₂ in chamber 5.

The volume of degassing chamber 5, into which the solvent is guided, is sufficiently large in order to ensure a long T₁, relaxation time of the hyperpolarized nuclei. The volume is dimensioned such that a gas pressure of about 2 bar is set.

Following degassing in chamber 5, the solution and degassing process may be repeated, as represented in FIG. 2 by continuous lines. For this, starting from chamber 5 at the end of the first enrichment stage, the gas with the hyperpolarized atomic nuclei is guided into chamber 6, where it is cooled to the temperature T₁. Following enrichment in the solvent, the gas is conducted into chamber 8 with a temperature of T₂ for degassing. Chamber 8, again, has a sufficient volume.

Chamber 6 is provided with a cooling unit 7 for cooling the solvent, as is chamber 2.

The respective temperatures T₁ and T₂ in chambers 2, 6, as well as 5, 8, may but need not be identical. Instead, the chambers may be provided with heating apparatuses and means for producing ultrasound, depending on the actual application. The degassed hyperpolarized atomic nuclei are guided from chamber B into a tank 9.

The inner walls of the tank 9 are lined with PFA or monochlorosilane in order to prolong the relaxation time of the hyperpolarized atomic nuclei. Basically, all chambers and connecting lines of the apparatus may be designed like this. The solvent is fed into a waste container 14 and disposed of, or stored temporarily for reuse in a container 10. The transport may be controlled by the gas pressure by exposing the solvent to pressure compensation, e.g. in container 10. The waste container 14 is arranged behind either chambers 5 or 8 for degassing. Subsequently, the solvent is returned through a cooling coil via connecting line 12 to chamber 2.

A storage tank 13 containing solvent is arranged before the cooling coil 11, so that spent solvent is replaced and may be pre-cooled to the intended temperature T₁.

The complete process may be controlled continuously or semi-continuously via the pressure tank 9 and pressure compensation container 10.

The process may be controlled at least partially via the drawn valves. Additional valves not shown may be arranged, e.g. behind chamber 8 before the branching off after waste container 14. The process may also be performed in one step, as indicated in FIG. by the dotted lines. Then, valves that are not shown ensure that the solvent released from chamber 5 may be fed into the pressure compensation container 10, or else an equivalent three-way valve may be inserted before the pressure compensation container.

To store the gas in tank 9, a magnet field below 0.01 T need only be provided, whereby Helmholt coils are appropriately arranged as a part of the apparatus.

The following ¹²⁹Xe densities in the solvent (toluol/ethanol) may be set at different temperatures:

| | Stage 1 | Stage 2 |
|---|---|---|
| | (Chamber 2) | (Chamber 6) |
| T = 240°K, | pXe, Sol −0.7 bar | ˜3.5 bar |
| T = 200°K, | pXe, Sol −2.1 bar | ˜10 bar |
| T = 180°K, | pXe, Sol −7.0 bar | ˜20 bar |

Similar values are obtained for pentane, acetone, methanol and other solvents.

Naturally, another valuable gas may be enriched, e.g. ¹³C, using such an apparatus.

1. A method of enriching a component of a gas mixture, characterized in that the component flowing in the gas mixture is dissolved in a solvent cooled to below 293° K.

2. The method according to the previous claim, characterized in that hyperpolarized atomic nuclei are enriched.

3. The method according to claim 1, characterized in that an organic hydrocarbon is used as solvent.

4. The method according to claim 1, characterized in that a solvent with an Ostwald coefficient of at least 2 is used for the hyperpolarized atomic nuclei or the component to be enriched.

5. The method according to claim 1, characterized in that ethanol, toluol, benzene, olive oil, butanol, pentane, methanol and/or acetone are used as solvent.

6. The method according to claim 1, wherein ¹²⁹Xe or ¹³C are enriched.

7. The method according to claim 1, characterized in that a deuterized solvent is used.

8. The method according to claim 1, wherein during enrichment, the cooled solvent is exposed to a maximum magnetic field of about 0.01 to 0.04 Tesla.

9. The method according to claim 1, wherein a temperature from 10 to 180° K of the cooled solvent is used.

10. The method according to claim 1, wherein degassing from the solvent is done following solution of the component or the hyperpolarized atomic nuclei.

11. The method according to the previous claim, wherein the solvent prior to degassing is guided into a chamber for degassing.

12. The method according to claim 1, characterized in that the steps for dissolving and degassing the components or the hyperpolarized atomic nuclei in and from the cooled solvent are repeated at least once.

13. The method according to claim 1, wherein a lowering of the melting point of the solvent occurs by dissolving the component to be enriched.

14. An apparatus for carrying out claim 1, comprising at least a chamber with means for degassing an enriched component.

15. The apparatus according to claim 14, comprising at least a chamber with a cooling apparatus.

16. The apparatus according to claim 14, characterized by means for forming a maximum magnetic field of 0.04 T.

17. The apparatus according to claim 16, comprising at least a Heimholtz coil and/or a permanent magnet.

18. The apparatus according to claim 14, characterized by a tank for the enriched component connected with the degassing chamber.

19. The apparatus according to claim 14, characterized in that the inner walls of the chambers, tank or other connecting lines comprise especially deuterized monochlorosilane and/or PFA.

20. The use of solvents cooled to below 293° K for enrichment, storage and/or transport of hyperpolarized atomic nuclei or ¹³C.

21. The use according to the previous claim 20, characterized by ethanol, toluol, benzene, olive oil, butanol, pentane, methanol and/or acetone as solvent.

22. The use according to claim 20, characterized by a lipophilic hydrocarbon as solvent.

23. The use according to claim 20, characterized by a deuterized solvent.

24. A solvent in which hyperpolarized atomic nuclei or ¹³C are dissolved, characterized in that the solvent has a temperature of below 293° K.

25. The solvent according the previous claim 24, characterized by ethanol, toluol, benzene, olive oil, butanol, pentane, methanol and/or acetone as solvent.

26. The solvent according to claim 24, haracterized in that the solvent is deuterized.

27. The solvent according to claim 24, characterized by ¹²⁹Xe as a hyperpolarized noble gas.

28. The solvent according to claim 24, characterized in that its melting point is lowered by dissolving hyperpolarized atomic nuclei or ¹³C.

29. Contrast agents comprising a solvent with hyperpolarized atomic nuclei according to claim 24.
